(19) United States
(12) Patent Application Publication
(10) Pub. No.: US 2007/0198045 A1
(43) Pub. Date: Aug. 23, 2007

(54) System to Facilitate the Use of a Surgical Instrument

(76) Inventors: John Morton, Palo Alto, CA (US); Jessica Connor, Palo Alto, CA (US); Gary Binyamin, Palo Alto, CA (US)

Correspondence Address: SCHOX PLC, 209 N. MAIN STREET #200, ANN ARBOR, MI, 48104, US

(21) Appl. No.: 11/677,514

(22) Filed: Feb. 21, 2007

Related U.S. Application Data
(60) Provisional application No. 60/775,391, filed on Feb. 21, 2006.

(51) Int. Cl. A61M 29/00

(52) U.S. Cl. 606/191

(57) ABSTRACT

The dilating device of the preferred embodiments includes a three dimensional dilating surface that functions to dilate an orifice, an alignment surface that functions to align and center the dilating device to the orifice, a coupling element that functions to couple the dilating device to the surgical instrument, a removal element that functions to facilitate the removal of the dilating device from the surgical site such as a hole and/or a grasping surface. The dilating device is preferably designed to dilate an orifice to the size of a surgical instrument, thereby facilitating the use of a surgical instrument during surgery and, more specifically, facilitating the use of a circular stapler in laparoscopic surgery. The dilating device, however, may be alternatively used in any suitable environment and for any suitable reason.

### CROSS-REFERENCE TO RELATED APPLICATIONS

This application claims the benefit of U.S. Provisional Application No. 60/775,391, filed 21 Feb. 2006 and entitled “A System to Facilitate the Use of a Surgical Instrument,” which is incorporated in its entirety by this reference.

### TECHNICAL FIELD

This invention relates generally to the surgical instrument field, and more specifically to an improved dilating device to facilitate the insertion of surgical instruments through natural and surgically created orifices.

### BACKGROUND

During surgery, surgical instruments that are inserted into the body through natural or surgically created orifices may be far too large to be inserted a-traumatically. This occurs in many surgeries performed in several clinical areas. For example, in Bariatric surgery and more specifically during a roux-en-y gastric bypass (RYGB) procedure, a surgeon will insert a large circular stapler through a trocar port site in the abdomen. Typically, the trocar port sites are 5 to 10 mm in diameter while the circular stapler is on the order of 25 mm in diameter. Conventionally, the surgeon is required to enlarge the trocar port site and use a great deal of force to insert the instrument through the wound. This can be dangerous both for the surgeon and for the patient. Thus, there is a need in the surgical instrument field for a device to dilate the orifice to the size of the instrument to be inserted. This invention provides such a new and useful dilating device.

### BRIEF DESCRIPTION OF THE FIGURES

FIGS. 1 and 2 are schematic drawings of the device of the first preferred embodiment of the invention.

FIGS. 3, 4, and 5 are schematic drawings of a second preferred embodiment of the invention.

### DESCRIPTION OF THE PREFERRED EMBODIMENTS

The following description of preferred embodiments of the invention is not intended to limit the invention to these embodiments, but rather to enable any person skilled in the art to make and use this invention.

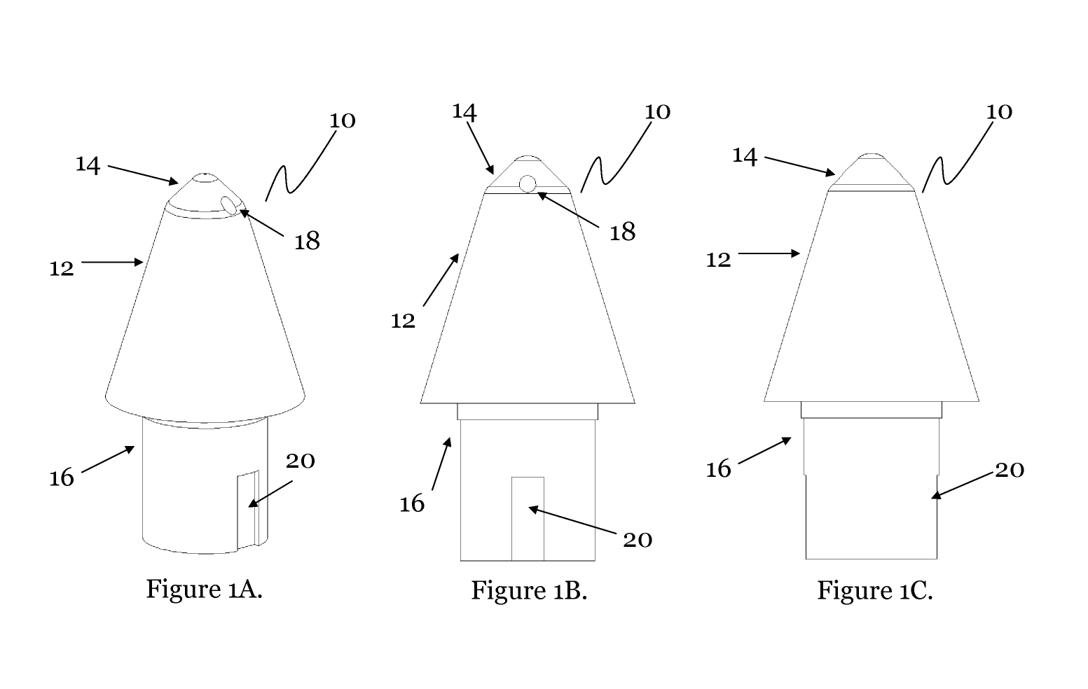

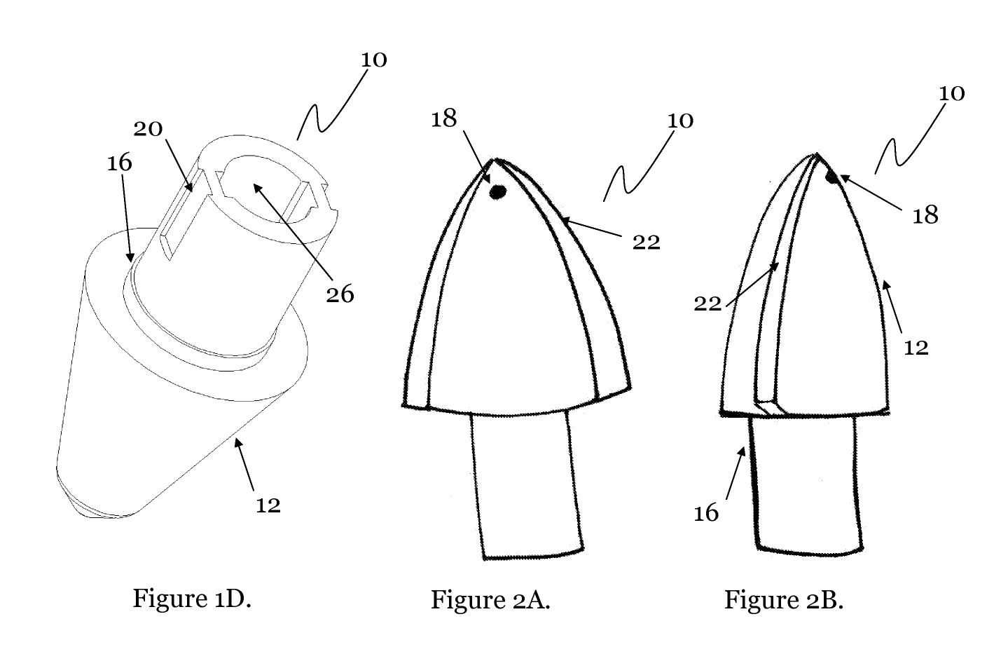

As shown in FIGS. 1 and 2, the dilating device 10 of a first preferred embodiment includes a three dimensional dilating surface 12 that functions to dilate an orifice, an alignment surface 14 that functions to align and center the dilating device 10 to the orifice, a coupling element 16 that functions to couple the dilating device 10 to the surgical instrument, a removal element 30 that functions to facilitate the removal of the dilating device 10 from the surgical site such as a hole 18 and/or a grasping surface 20. The dilating device 10 is preferably designed to dilate an orifice to the size of a surgical instrument, thereby facilitating the use of a surgical instrument during surgery and, more specifically, facilitating the use of a circular stapler in laparoscopic surgery. The dilating device 10, however, may be alternatively used in any suitable environment and for any suitable reason.

The three dimensional dilating surface 12 of the preferred embodiments functions to dilate an orifice. The dilating surface 12 is preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. The dilating surface 12 is preferably a smooth surface but may alternatively be any suitable surface such as one with bumps, ridges, a dilating edge 22 (as shown in FIG. 2), threads, or any other suitable surface to dilate an orifice.

The dilating surface 12 is preferably one of several variations. In a first variation, as shown in FIG. 1, the dilating surface 12 is a conical geometry preferably with an opening angle (vertex angle) less than 45 degrees but alternatively with any suitable opening angle to minimize damage to the tissue. The conical dilating surface preferably has a base diameter that is greater or equal to the outer diameter of the surgical instrument. The dilating surface 12 is preferably a conical geometry as it has been shown to create an abdominal wall defect up to 50% smaller than a pyramidal geometry. Additionally, there is a lower risk of inadvertent injury with a conical geometry and less bleeding due to the fact that the conical geometry tends to separate the tissue with little damage rather than to lacerate the tissue and vessels. The dilating surface 12 may alternatively be a pyramidal geometry, a parabolic geometry as shown in FIG. 2, a semi-hyperbolic geometry, a semi-elliptical geometry, a semi-spherical geometry, or any other suitable geometry to dilate an orifice.

Although the dilating surface 12 is preferably one of these multiple variations, the dilating surface 12 may be any suitable material, surface, geometry, and combination of elements to dilate an orifice while causing minimal damage to the tissue.

As shown in FIG. 1, the alignment surface 14 of the first preferred embodiment functions to align and center the dilating device 10 to the orifice and provide a blunt tip so as to not damage the vital structures within the patient upon insertion of the dilating device 10. The top portion of the dilating device 10, from the tip to the point where the diameter of the dilating device 10 is equal to the orifice, is the alignment surface 14. The alignment surface 14 is preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. The alignment surface 14 is preferably a smooth surface but may alternatively be any suitable surface such as one with bumps, ridges, a dilating edge 22 (as shown in FIG. 2), threads, or any other suitable surface to align and center the dilating device 10 to the orifice and provide a blunt tip so as to not damage the vital structures within the patient upon insertion of the dilating device 10.

The alignment surface 14 is preferably one of several variations. In a first variation, as shown in FIG. 1, the alignment surface 14 is a conical geometry preferably with an opening angle greater than the opening angle of the dilating surface 12 but alternatively with any suitable opening angle to align and center the dilating device 10 to the orifice and provide a blunt tip so as to not damage the vital structures within the patient upon insertion of the dilating device 10. In a second variation, as shown in FIG. 2, the dilating surface 12 is preferably a parabolic geometry but may alternatively be any suitable geometry that functions to align and center the dilating device 10 to the orifice and provide a blunt tip so as to not damage the vital structures within the patient upon insertion of the dilating device 10 such as an elliptical or spherical geometry. Although the alignment surface 14 is preferably one of these multiple variations, the alignment surface 14 may be any suitable material, surface, geometry, and combination of elements to align and center the dilating device 10 to the orifice and provide a blunt tip so as to not damage the vital structures within the patient upon insertion of the dilating device 10.

As shown in FIGS. 1 and 2, the coupling element 16 of the first preferred embodiment functions to couple the dilating device 10 to the surgical instrument that is to be inserted into the orifice. The coupling element 16 is preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. The coupling element 16 is preferably a smooth surface but may alternatively be any suitable surface such as one with bumps, ridges, threads, or any other suitable surface to couple the dilating device 10 to the surgical instrument.

The coupling element 16 is preferably one of multiple variations. In a first variation, as shown in FIGS. 1A, 1B, and 1C, the coupling element 16 comprises a plurality of cylinders of varying diameters and thicknesses as to correspond to the geometry of the end of the surgical instrument. The geometry of the coupling element 16 in the first variation preferably matches that of an ENDOPATH ILS Endoscopic Curved Intraluminal Stapler ECS25 (Ethicon Endo-Surgery, Cincinnati, Ohio) but may alternatively match the geometry of any suitable circular stapler (from other manufacturers such as Tyco and US Surgical) or any other suitable surgical instrument. In the first variation, the dilating device 10 will press fit into the end of the stapler and remain fixed in place until the spike is advanced out of the end of the stapler, pushing the dilating device 10 out of the end out the stapler.

In a second variation, as shown in FIG. 1D, the coupling element 16 may further comprise a plug 26 that functions to receive and hold the spike of the circular stapler. The plug 26 is preferably made out of silicone but may alternatively be made out of any suitable material that will allow the shaft to puncture the material and then will hold the shaft in place. In a third variation, the coupling element 16 is an attachment mechanism that will removably attach to the surgical instrument. To remove the dilating device 10 in order to use the surgical instrument, the attachment mechanism may open, pop off, or slide down the shaft of the instrument to move out of the way of the functioning end of the instrument. In the case of the later, the dilating surface may comprise a plurality of dilating elements 24 that will move from the closed position to the open position as the coupling element 16 slides down the shaft of the surgical instrument.

In a fourth variation, the coupling element 16 may be a shaft that functions to couple the dilating device 10 to the surgical instrument in the same manner that the stem of the anvil of the stapler couples to the stapler (in the case that the surgical instrument is a circular stapler). In this variation, the shaft is preferably made to match the geometry of the stem of the anvil and is preferably a rigid material such as metal, plastic, or any other suitable material. The shaft will mechanically couple to the stapler through a snap fit or a press fit. Although the coupling element 16 is preferably one of these multiple variations, the coupling element 16 may be any suitable material, geometry, include any suitable connection mechanism, and combination of elements to couple the dilating device 10 to the surgical instrument.

The removal element 30 of the first preferred embodiment functions to facilitate the removal of the dilating device 10 from the surgical site and is one of multiple variations. In a first variation, the removal element 30 is a hole 18. The hole 18, as shown in FIGS. 1A, 1B, and 2, of the first preferred embodiment functions to provide a location where a loop or hook may be attached. The loop or hook functions to allow the user to grasp the dilating device 10 by the loop or hook, pull up on the loop or hook, and thus remove the dilating device 10, in the tip-first orientation, through an orifice in the opposite direction. The loop or hook is preferably a suture or a string that can be tied though the hole 18 prior to the insertion of the dilating device 10. The loop or hook may alternatively be any suitable handle, chain, linkage, or mechanism to facilitate the removal of the dilating device 10.

As shown in FIG. 1, in a second variation, the removal element 30 is a grasping surface 20. The grasping surface 20 functions to provide a surface ideally suited to be grasped by suitable surgical instruments such as surgical graspers. This will allow the user to manipulate the dilating device 10 to move and remove the dilating device 10 as desired. The grasping surface 20 is preferably located on a non-dilating surface of the dilating device 10 but may alternatively be located on any suitable surface. The grasping surface 20 is of a thickness adapted to allow surgical instruments such as surgical graspers, with limited ranges of motion to grasp the surface. The grasping surface 20 is preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. The grasping surface 20 has a surface texture ideally suited to be grasped by suitable surgical instruments such as surgical graspers. The surface texture is preferably rough such as with bumps, ridges, edges, or threads to facilitate gripping, but may alternatively be a smooth surface or any other suitable surface or material to provide a surface ideally suited to be grasped by suitable surgical instruments such as surgical graspers.

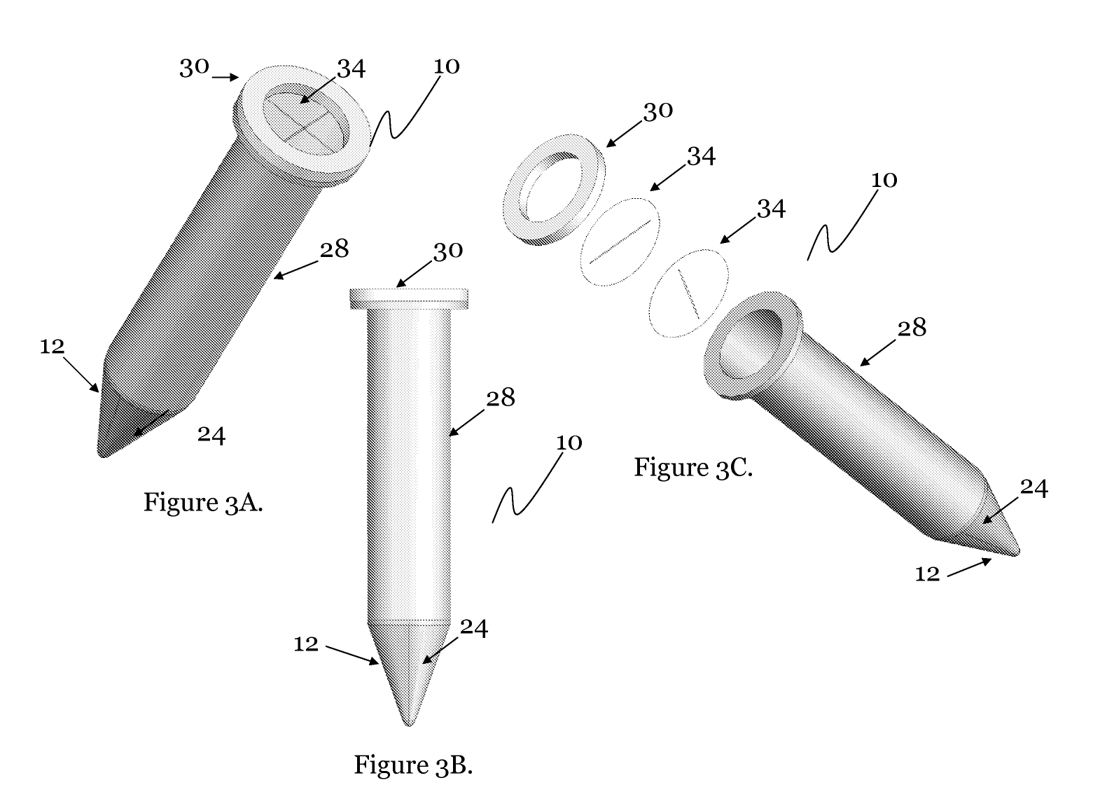

As shown in FIGS. 3, 4, and 5, the dilating device 10 of a second preferred embodiment includes a three dimensional dilating surface 12 that functions to dilate an orifice, a plurality of dilator elements 24, a sleeve element 28 that functions to protect the orifice from the surgical instrument and from infection, a coupling element 16 that functions to couple the dilating device 10 to the surgical instrument, a removal element 30 that functions to facilitate the removal of the dilating device 10 from the surgical site and to prevent the dilating device 10 from entering, in its entirety, through the orifice. The dilating device 10 may be inserted by grasping the dilating device 10 directly, or the surgical instrument may be inserted into the dilating device 10 and then used as a handle to insert the dilating device 10 into the orifice. Once inserted, the dilating device 10 of the second preferred embodiment may be used as a receptacle to hold tissue specimen or other items to be removed during the course of a surgery. The dilating device 10 is preferably designed to facilitate the use of a surgical instrument and, more specifically, to facilitate the use of a circular stapler in laparoscopic surgery. The dilating device 10, however, may be alternatively used in any suitable environment and for any suitable reason.

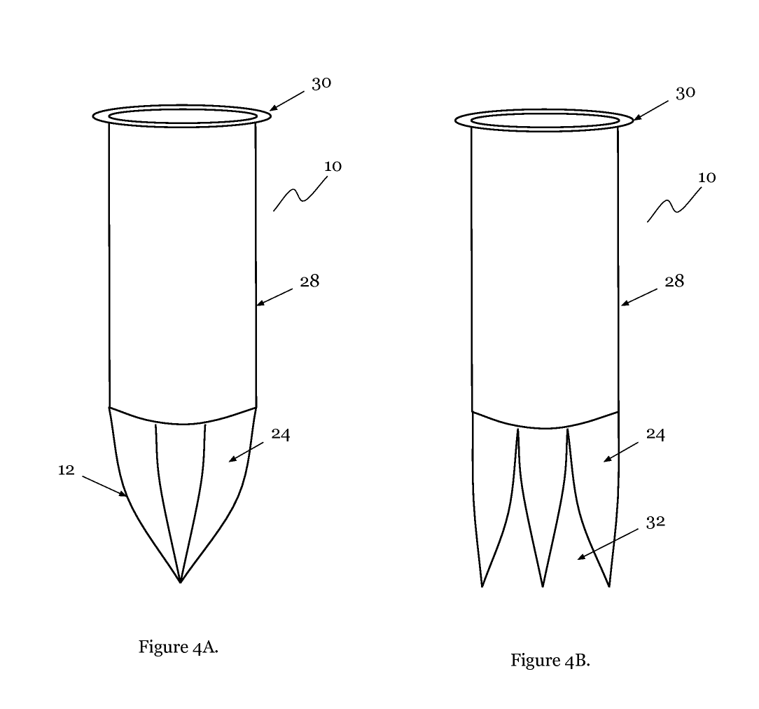

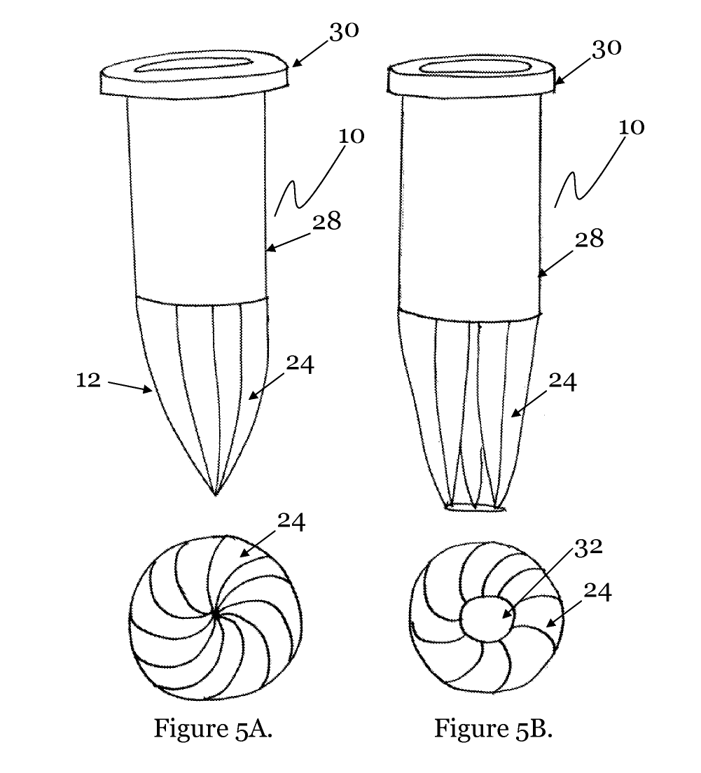

As shown in FIGS. 3, 4, and 5, the three dimensional dilating surface 12 of the second preferred embodiment comprises a plurality of dilator elements 24. The dilator elements 24 are preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. The dilator elements 24 preferably include at least two surfaces, one being an exterior dilating surface and the other an interior surface. The dilator elements 24 have a closed position, as shown in FIGS. 4A and 5A, and an open position, as shown in FIGS. 4B and 5B. While in the closed position, the dilator elements 24 form the dilating surface 12 and are rigid in the axial direction. The dilator elements 24 will resist deflection and will dilate the orifice. Further, in the closed position, the pneumoperitoneum and insulflation level will be maintained by preventing leakage of the gas or liquid from the peritoneal, thoracic, or other suitable cavity. In the open position, the dilator elements 24 will open and provide an opening 32 for the insertion of the surgical instrument. The opening 32 may be any suitable size to allow for the passage of any suitable surgical instrument. For a given dilating device 10, there may be pre-set sizes for the openings 32, such that certain objects of certain sizes may be passed through.

In a first variation, as shown in FIG. 4, the dilator elements 24 will fold open from a hinge or joint. The hinge is preferably a living hinge but may alternatively be any standard mechanical hinge to allow the dilator elements 24 to fold open. In this embodiment, the coupling element 16 is a point or ledge on the interior surfaces of the dilating elements 24, where the surgical instrument is coupled to the dilating device 10. The dilator elements 24 will fold open once pressure is exerted by the surgical instrument on the coupling element 16 as it is inserted through the dilating device 10. Alternatively, the dilator elements 24 will fold open when a button is pressed, a switch is flipped, or when any suitable mechanism is activated.

In a second variation, as shown in FIG. 5, the dilator elements will slide circumferentially over one another to open. The opening mechanism in this variation preferably resembles that of a shutter where the dilating elements 24 are turned and twisted in such a manner that they provide an opening 32, but may alternatively use any suitable mechanism to open the dilating elements 24 to provide an opening 32. Although the dilating elements 24 are preferably one of these multiple variations, the dilating elements 24 may open in any suitable mechanism such as rolling up, deflating, folding up, sliding up into the sleeve element 28, or breaking off.

The sleeve element 28 of a second preferred embodiment functions to protect the orifice from the surgical instrument and infection. The sleeve element 28 is preferably a smooth surface but may alternatively be any suitable surface such as one with bumps, ridges, threads, or any other suitable surface to protect the orifice and aid in holding the dilating device 10 in place. The sleeve element 28 is long enough to go through the entire abdominal wall, or any other suitable anatomical location, but will not prevent the user from easily manipulating the surgical instrument within the dilating device 10. The sleeve element 28, in a first variation, as shown in FIGS. 3 and 5, is preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. In a second variation, as shown in FIG. 4, the sleeve element 28 is made from a flexible material such as a plastic, but may alternatively be made of any suitable flexible material.

The removal element 30 of the second preferred embodiment functions to facilitate the removal of the dilating device 10 from the surgical site and to prevent the dilating device 10 from entering, in its entirety, through the orifice. The removal element 30 comprises a surface that is larger than the orifice and that will rest on the outside of the patient and an opening through which objects may be inserted or removed. The removal element 30 is preferably made from a rigid material such as plastic or metal. The material may be a rigid material that is capable of becoming flexible or changing shape due to the application of electricity or heat. Alternatively the material may be a flexible material that is made rigid by applying electricity or heat or by filling the material with a liquid or gas. The material may alternatively be flexible or partially rigid such as a gel or flexible plastic such as silicone. The removal element 30 may alternatively be made out of a flexible material such that it may move with the patient and be adjustable and movable.

The removal element 30 may further comprise a diaphragm element 34 that functions to maintain the pneumoperitoneum and insulflation level by preventing leakage of the gas or liquid from the peritoneal, thoracic, or other suitable cavity. The diaphragm element 34 will maintain the pneumoperitoneum and insulflation level when the dilating elements 24 are both in the opened and closed position. The diaphragm element 34 is preferably a flexible material, but may alternatively be a rigid material that will fold or bend to allow the passage of the surgical instrument or other object. The removal element 30 may comprise a plurality of diaphragm elements 34.

Although omitted for conciseness, the preferred embodiments include every combination and permutation of the various dilating surfaces 12, the various alignment surfaces 14, the various coupling elements 16, the various removal elements 30, the various holes 18, the various grasping surfaces 20, the various dilating edges 22, the various dilating elements 24, the various plugs 26, the various sleeve elements 28, the various openings 32, and the various diaphragm elements 34.

As a person skilled in the art will recognize from the previous detailed description and from the figures and claims, modifications and changes can be made to the preferred embodiments of the invention without departing from the scope of this invention.

We claim:

1. A dilating device for dilating an orifice to the size of a surgical instrument, wherein the surgical instrument includes an end portion with an inner diameter and an outer diameter, the dilating device comprising:
a three dimensional dilating surface adapted to dilate an orifice to the size of the surgical instrument;
a coupling element adapted to couple the dilating device to the end portion of the surgical instrument; and
a removal element adapted to facilitate the removal of the dilating device.

2. The dilating device of claim 1 wherein the dilating surface is conical with an opening angle less than 45 degrees and a base diameter at least equal to the outer diameter of the surgical instrument.

3. The dilating device of claim 1 wherein the dilating surface has a geometry that is selected from the group consisting essentially of pyramidal, parabolic, semi-hyperbolic, semi-elliptical, and semi-spherical.

4. The dilating device of claim 1 wherein the dilating surface is a rigid surface.

5. The dilating device of claim 1 wherein the dilating surface includes a flexible membrane wherein the flexible membrane is adapted to become rigid by filling with at least one of a liquid and gas.

6. The dilating device of claim 1 wherein the dilating surface further includes a dilating edge adapted to aid in the dilation of an orifice wherein the dilating edge is selected from the group consisting essentially of bumps, ridges, threads, and combinations thereof.

7. The dilating device of claim 1 wherein the dilating surface further includes a three dimensional alignment surface, wherein the alignment surface is the top portion of the dilating surface and is adapted to align the dilating device with the orifice and the alignment surface has an opening angle greater than the opening angle of the dilating surface such that it is adapted to create a blunt tip on the dilating device.

8. The dilating device of claim 1 wherein the coupling element has a cylindrical geometry, wherein the cylindrical geometry includes at least one cylinder with a diameter that is smaller than the inner diameter of the surgical instrument such that coupling element couples the dilating device to the inner diameter of the surgical instrument.

9. The dilating device of claim 1 wherein the coupling element includes a piercable plug adapted to receive and removably hold a portion of the surgical instrument.

10. The dilating device of claim 1 wherein the coupling element is an attachment mechanism adapted to removably attach to the outer diameter of a surgical instrument.

11. The dilating device of claim 1 wherein the removal element includes a hole defined by at least one of the dilating surface and the alignment surface.

12. The dilating device of claim 1 wherein the removal element includes a grasping surface, wherein the grasping surface has a thickness and a texture adapted to be grasped by surgical instruments.

13. The dilating device of claim 1 wherein the dilating surface further includes a plurality of dilator elements, wherein the dilator elements include an exterior dilating surface and an interior surface.

14. The dilating device of claim 13 wherein the dilating elements are adapted to operate in the following modes:
closed mode, wherein the dilating elements collectively form the dilating surface and are adapted to be rigid in the axial direction; and
open mode, wherein the dilating elements define an opening adapted to allow the passage of objects.

15. The dilating device of claim 14 wherein the dilator elements are adapted to transition from the closed mode to the open mode by folding open at a hinge.

16. The dilating device of claim 14 wherein the dilator elements are adapted to transition from the closed mode to the open mode by sliding circumferentially over one another.

17. The dilating device of claim 14 wherein the internal surfaces of the dilator elements in closed mode define the coupling element as a ledge, wherein the dilator elements are adapted to transition from the closed mode to the open mode when pressure is exerted by the surgical instrument on the coupling element.

18. The dilating device of claim 13 wherein the removal element is larger than the orifice and is adapted to not pass through the orifice with the surgical instrument.

19. The dilating device of claim 18 wherein the removal element further includes a diaphragm element adapted to prevent the passage of gas through the orifice.

20. The dilating device of claim 13 further including a sleeve element adapted to line and protect the orifice.
